(19) United States
(12) Patent Application Publication
(10) Pub. No.: US 2005/0279663 A1
(43) Pub. Date: Dec. 22, 2005

(54) Toothbrush holder

(76) Inventor: Angelo Citrigno, Thorold (CA)

Correspondence Address: GOWLING, LAFLEUR HENDERSON LLP, ONE MAIN STREET WEST, HAMILTON, ON, L8P 4Z5, CA

(21) Appl. No.: 10/872,370

(22) Filed: Jun. 22, 2004

(51) Int. Cl. B65D 81/24; B65D 83/10; A47B 81/02

(52) U.S. Cl. 206/362.1; 211/66

(57) ABSTRACT

The present invention provides a toothbrush holder assembly for storing and sanitizing a toothbrush. The assembly comprises a holder compartment and a support platform. In use, a disinfectant solution is poured into the holder compartment and the toothbrush head is immersed in the disinfectant solution. The assembly can be adapted for mounting on a wall or on a horizontal surface.

### FIELD OF INVENTION

The present invention relates to a container system for sanitizing and storing toothbrushes.

### BACKGROUND OF THE INVENTION

It is well known that the human mouth is home to many different types of microorganisms. It is also well known that these microorganisms may contribute to periodontal disease, halitosis and other types of infection. It is recommended to brush and floss carefully to remove these germs. In the process, however, bacteria are transferred from the user's mouth and collect on the bristles of the toothbrush head. No matter how thoroughly the toothbrush is rinsed, it is virtually impossible to remove them all. In addition, the moist bristles of the toothbrush provide a fertile environment for colonization by microorganisms that are present in aerosols in the bathroom. Most of us would never consider eating or storing food in the bathroom, yet we think nothing of putting a toothbrush that has been stored in a less than hygienic environment, such as a bathroom counter, into our mouths. This issue is particularly relevant after a pathogenic infection. Germs from the mouth are deposited on the toothbrush. This can lead to re-infection with a pathogenic microorganism. In addition, toothbrushes are often stored in racks close together. This means that germs from one toothbrush can be transferred to another. Thus, a typical toothbrush can harbor microorganisms from the mouth and from the environment.

The problem of contaminated toothbrushes has long been recognized and numerous attempts have been made to provide a means for the sanitary storage of toothbrushes. Various types of toothbrush holders are described, for example, in U.S. Pat. Nos. 4,570,652; 4,915,219; 4,995,509; 4,997,629; 5,107,987; 5,377,824; 5,566,823; 5,690,214; 5,922,292; 6,135,279 and 6,601,699. There are several problems associated with the prior art devices. In some cases the devices are complex to manufacture and therefore costly. In other cases, a plurality of toothbrushes are stored within a single reservoir of disinfectant providing an opportunity for cross-infection.

U.S. Pat. No. 6,119,854 discloses a sanitary toothbrush storage unit that includes a divider to provide separate compartments for each toothbrush. However, the compartments form a unitary device whereby it is necessary to change the sanitizing fluid in both compartments at the same time.

U.S. Pat. No. 6,360,884 discloses another type of toothbrush holder that includes means for connecting a plurality of holders together. One problem with this type of holder is that the entire toothbrush including the handle is put into a closed holder. This necessitates reaching into the container to retrieve the toothbrush and introduces the possibility of additional contamination from the handle.

While there have been many attempts to provide means of storing a toothbrush hygienically, there remained an unmet need for an efficient, cost-effective toothbrush holder system that could be used to disinfect a toothbrush between uses.

### SUMMARY OF THE INVENTION

The present invention relates to a device for the sanitary storage of a toothbrush.

It is an object of the invention to provide an improved toothbrush holder assembly.

According to one aspect of the present invention, there is provided a holder system for at least one toothbrush. The holder system comprises a toothbrush holder compartment and a holder platform. The holder platform can be either surface or wall mounted. The toothbrush holder compartment is adapted to hold an amount of a disinfectant solution, such as a mouthwash solution. Preferably, the toothbrush holder compartment is sized to hold the head/bristle portion of a toothbrush with a minimum of excess volume so as to require a minimum amount of disinfecting solution to cover the bristle portion. The system is designed so that each toothbrush is stored in a separate compartment to reduce the risk of cross-contamination.

The toothbrush holder assembly of the present invention typically includes a plurality of toothbrush compartments mounted on a holder platform. Multiple units of the system can be used to adjust to the number of required compartments. For example, if a basic system is provided with three toothbrush compartments, two systems can be used to provide six compartments. A basic system preferably comprises from two to six toothbrush holder compartments.

The toothbrush compartments and platform are preferably a clear polyacrylic material. It is clearly apparent, however, that any material that is sufficiently rigid and non-porous could be used in the manufacture of the device.

A preferred platform according to the present invention is adapted to be mounted on a vertical surface. The vertical surface could be a wall or a cupboard door. In one preferred embodiment, the platform comprises a planar backsheet, a base and at least one receptacle for receiving a toothbrush compartment. The receptacle may be open or closed ended.

In another embodiment, the receptacle is sized to frictionally engage the toothbrush holder compartment.

In another aspect of the invention (not shown), the platform is adapted for placement on a horizontal surface such as a counter top or a shelf. In one embodiment, the holder comprises a planar backsheet and a base panel that together form an “L” structure. Receptacles for receiving a toothbrush compartment are mounted on the backsheet and the base panel rests on a horizontal surface.

In another embodiment, the holder system comprises a base panel on which receptacles for receiving toothbrush compartments are directly mounted.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1 shows a perspective view of a toothbrush holder assembly in accordance with an embodiment of the present invention

FIG. 2A shows a perspective view of a toothbrush compartment of the system of FIG. 1;

FIG. 2B shows a side view of the toothbrush compartment of FIG. 2A;

FIG. 2C is an end view of the toothbrush compartment of FIG. 2B;

FIG. 3A is a side view of a support platform of the toothbrush holder assembly of the invention;

FIG. 3B is a front view of the support platform of FIG. 3A; and

FIG. 3C is a top view of the support platform of FIG. 3B.

### DETAILED DESCRIPTION

Toothbrushes provide an environment for the growth of various different types of micro-organisms, including bacteria, viruses and fungi. Micro-organisms on a toothbrush can result in recurrent infections, especially infections of the mouth and throat. There is a need, therefore, for a means of storing a toothbrush that disinfects it and prevents it from becoming contaminated from the environment. The present invention addresses this need by providing a novel toothbrush holder assembly.

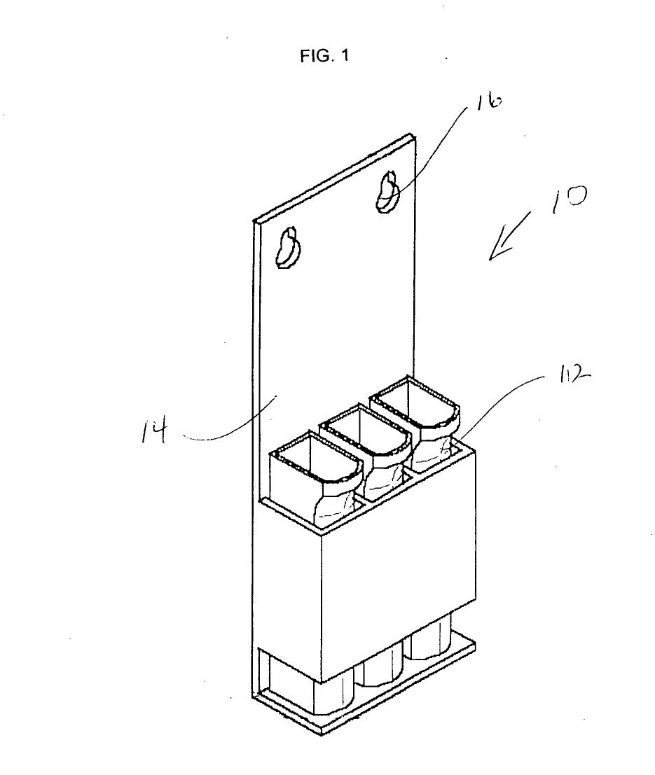

In one preferred embodiment, as shown in FIG. 1, the toothbrush holder assembly 10 comprises at least one toothbrush holder compartment 12 and a platform 14 for holding the compartments. The system shown in FIG. 1 is adapted for mounting on a wall and includes apertures 16 for receiving a nail or screw. In another embodiment of a wall-mounted unit, the platform is adhesively mounted to the surface. Wall-mounted units can be affixed to a wall, a cupboard door, a drawer side or any other vertical surface. In another embodiment (not shown), a platform is provided that is adapted for resting on a horizontal surface.

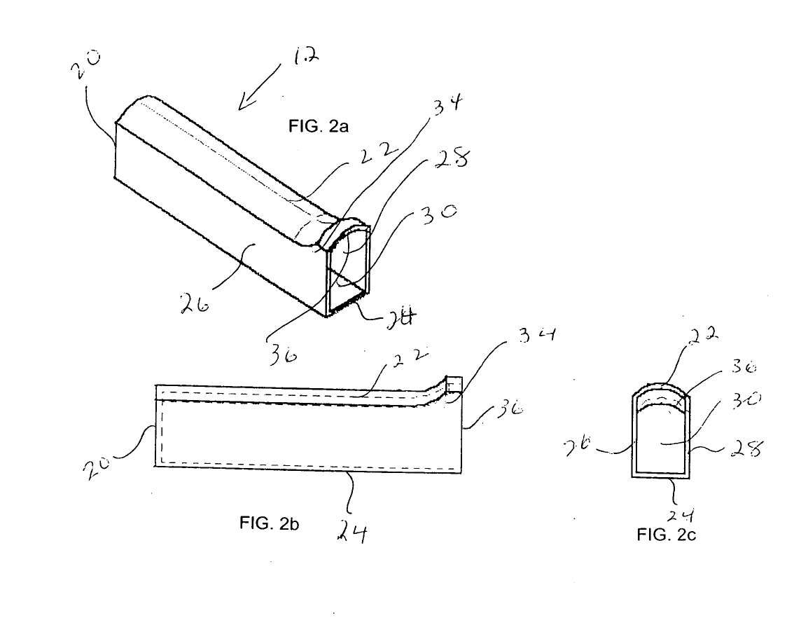

As shown in greater detail in FIG. 2, the toothbrush holder compartment 12 comprises a base 20, a front wall 22, a rear wall 24 and two side walls 26, 28 which combine to form a reservoir 30. FIG. 2A shows a perspective view of the compartment, FIG. 2B shows a side view and FIG. 2C shows a top view. The two side walls optionally include a flared end 34 at the open top end 36 of the compartment. In this way, the open end is slightly larger than the rest of the compartment to facilitate putting the brush in the compartment and reduce spraying of moisture. The front wall 22 of the compartment may be slightly convex to form a curved front surface. Although not shown in the figures, it is clearly apparent that the reservoir could also be formed by a continuous circular or oval wall.

In use, the reservoir is filled with a disinfectant solution to a volume sufficient to cover the head/bristle portion of the toothbrush. While the open end of the reservoir may be flared wider, the perimeter of the reservoir is generally slightly larger than the size of the head of a toothbrush. Because of this, the volume of disinfectant that is required to cover the bristles is minimized. Various types of disinfectants can be used. For example, an antiseptic mouthwash solution could be used or an antibiotic solution could be used.

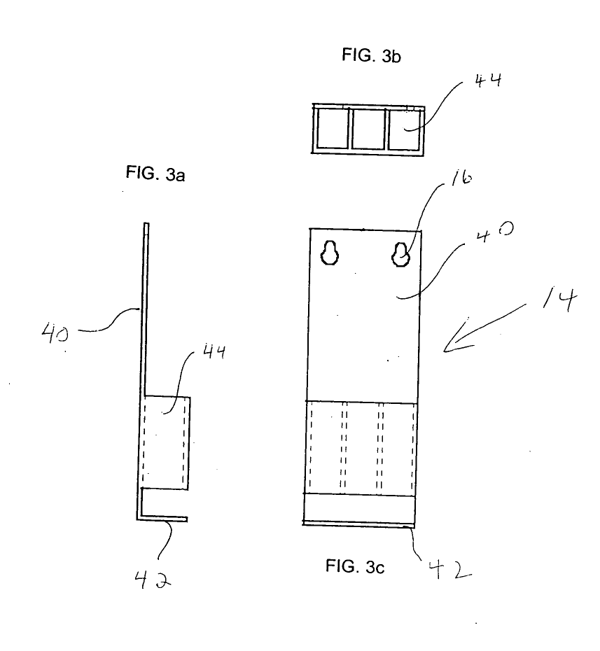

An exemplary embodiment of a support platform is shown in FIG. 3. The platform 14 comprises a planar back sheet 40, a base 42 and at least one receptacle 44 for receiving a toothbrush compartment. The receptacle is adapted to hold a toothbrush compartment. Although illustrated as an o pen ended rectangular structure that snugly holds the compartment in place, it is clearly apparent that the receptacle could have a closed bottom to hold the compartment by gravity. It is also apparent that the shape of the receptacle would be adapted according to the shape of the toothbrush compartment (i.e. square, rectangular, circular, bval, etc. . . . ).

The backsheet may include apertures 16 for receiving a nail or screw for mounting, as shown, or it may be adhesively adhered to a vertical surface. The platform is typically mounted on a wall or on the inside of a cupboard or drawer in a bathroom.

In another embodiment (not shown) a platform adapted for a horizontal surface is provided. The surface-mounted platform may be affixed to the surface or it may be free standing. In one embodiment of a platform adapted for a horizontal surface, the platform comprises a backsheet and a base. The receptacles are attached to the backsheet and the base is sufficiently large to provide stability. In another embodiment, the platform comprises a base on which receptacles for receiving the toothbrush compartments are mounted.

The present invention will be further illustrated in the following examples.

### EXAMPLES

### Example 1

### Construction of a Toothbrush Holder Assembly

A toothbrush compartment is constructed of clear plastic using a mold. The dimensions cited herein are approximate and encompass a variation of about 20%. The side walls of the compartment are about 8-12, preferably about 10, cm long. At the bottom of the compartment, the side walls are 1 to 4, preferably about 2.5, cm wide and at the top flared end, the side walls are about 3-6 cm wide. The walls are about 0.1 to 0.2 cm thick and the base is about 1 to 3, preferably about 2, cm wide.

A clear plastic platform is constructed. The backsheet is 15 to 30, preferably about 20, cm long. It is about 6 to 9, preferably about 7.5, cm wide. The platform is adapted to hold three toothbrush compartments and thus there are three receptacles for receiving the compartments. The receptacles are about 6-7 cm long.

For use, a toothbrush compartment is inserted into a receptacle and slides down to rest on the base.

### Example 2

### Method of Storing a Toothbrush

A toothbrush holder assembly as described in Example 1 is provided. At least one toothbrush compartment is held in a receptacle. A volume of about 1 cc to about 4 cc disinfectant mouthwash is put into the receptacle. After brushing, the toothbrush is rinsed and then inserted into the toothbrush compartment such that the bristle portion is covered by the disinfectant mouthwash. Every 24 hours, the disinfectant solution is emptied and refilled with fresh solution.

All citations are hereby incorporated by reference.

The present invention has been described with regard to one or more embodiments. However, it will be apparent to persons skilled in the art that a number of variations and modifications can be made without departing from the scope of the invention as defined in the claims.

1. A toothbrush holder assembly comprising at least one toothbrush holder compartment and a support platform for holding said toothbrush holder compartment.

2. A toothbrush holder assembly according to claim 1; wherein said support platform comprises:
i. a vertical backsheet;
ii. a horizontal base connected to said backsheet; and
iii. at least one receptacle mounted on the backsheet and adapted to receive a toothbrush holder compartment.

3. A toothbrush holder assembly according to claim 1, wherein the toothbrush holder compartment comprises a reservoir for holding a toothbrush, said reservoir having a front wall, a back wall, two side walls and a closed bottom.

4. A toothbrush holder assembly according to claim 1, wherein the toothbrush holder compartment is rectangular, circular or oval.

5. A toothbrush holder assembly according to claim 1, wherein the toothbrush holder compartment and the support platform are made of clear plastic.

6. A toothbrush holder assembly according to claim 1, wherein the support platform comprises a base and at least one receptacle mounted on said base for receiving at least one toothbrush holder compartment.

7. A method of storing a toothbrush, said method comprising a) providing a toothbrush holder assembly as defined in any one of claims 1 to 6; b) putting a disinfecting solution in the toothbrush holder compartment; c) storing a toothbrush in the compartment; and d) changing the disinfecting solution after every use of the toothbrush.
